(19) United States
(12) Patent Application Publication
(10) Pub. No.: US 2026/0363625 A1
(43) Pub. Date: Nov. 19, 2026

(54) ELIMINATION OF ARTEFACTS IN CRANIAL ACCELEROMETRY SIGNALS

(71) Applicants: Brainlab AG, Munich (DE); Brain-Pulse, Inc., Westchester, IL (US)

(72) Inventors: Nicolas MÜLLER, Munich (DE); Stefan HAGER, Munich (DE); Ray SCHUMACHER, Mountain View, CA (US); Dave M. COOK, Mountain View, CA (US); Lukas OTTER, Munich (DE)

(21) Appl. No.: 19/137,818

(22) PCT Filed: Dec. 19, 2023
(86) PCT No.: PCT/EP2023/000064

§ 371 Date: Jun. 11, 2025

(30) Foreign Application Priority Data
Dec. 19, 2022 (WO) PCT/EP2022/086728

(51) Int. Cl. A61B 5/00; A61B 3/113; A61B 5/0205; A61B 5/024; A61B 5/026; A61B 5/11

(52) U.S. Cl. CPC A61B 5/7203; A61B 5/0205; A61B 5/1114; A61B 5/7267; A61B 3/113; A61B 5/024; A61B 5/026; A61B 5/1124; A61B 5/4064; A61B 2560/0247; A61B 2562/0219

(57) ABSTRACT

Disclosed is a computer-implemented method of processing data acquired using acceleration sensors contacting an anatomical body part of a patient encompasses acquisition of acceleration measurement data via acceleration sensors, wherein possible artefacts within the acceleration signals are identified. Sections of the acceleration signals which contain identified artefacts are then disregarded in subsequent processing steps, or artefacts are removed therefrom, such that a comparison between artefact-free acceleration signals acquired from different subjects having a similar and/or dissimilar physiological status is possible.

### FIELD OF THE INVENTION

The present invention relates to a computer-implemented method of processing data acquired using acceleration sensors contacting an anatomical body part of a patient, a corresponding computer program, a computer-readable storage medium storing such a program and a computer executing the program, as well as a medical system comprising an electronic data storage device and the aforementioned computer.

### TECHNICAL BACKGROUND

Determination of brain anomalies such as a large vessel occlusion, aneurysm, or vasospasm is typically done using imaging modalities (e.g., CT, MRI, etc.) and other technical approaches such as EEG, Doppler sonography, cerebral ultrasound, or auscultation via microphones. Most of these approaches can only be performed in a clinical environment and thus there is a need of a mobile application for enabling an aid to diagnosis directly at the patient's site. Cranial accelerometry has already been described as a potential solution but comes with a sensitivity to interferences. With the proposed methods, this is addressed providing a user-friendly and reliable system to generate data suitable for analysis.

Acceleration sensors are highly sensitive and thus prone to artefacts. For a meaningful conclusion drawn from the cranial acceleration recording, high quality and mostly artefact-free data is required for analysis.

The present invention has the object of detecting and removing artefacts from acceleration signals, such that artefact-free data is available for analyzing these signals so as to reliably detect indicative features therein, which can then be used for classifying a subject/patient based on that indication.

The presented invention can be used for cranial accelerometry based triaging procedures of patients directly on location to prepare for execution of established triage methods such as CPSS (Cincinnati Prehospital Stroke Scale), RACE (Rapid Arterial Occlusion Evaluation), or LAMS (Los Angeles Motor Scale).

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

The disclosed computer-implemented method of processing data acquired using acceleration sensors contacting an anatomical body part of a patient encompasses acquisition of acceleration measurement data via acceleration sensors, wherein possible artefacts within the acceleration signals are identified. Sections of the acceleration signals which contain identified artefacts are then disregarded in subsequent processing steps, or artefacts are removed therefrom, such that a comparison between artefact-free acceleration signals acquired from different subjects having a similar and/or dissimilar physiological status is possible.

### GENERAL DESCRIPTION OF THE INVENTION

In this section, a description of the general features of the present invention is given for example by referring to possible embodiments of the invention.

In general, the invention reaches the aforementioned object by providing, in a first aspect, a computer-implemented medical method of processing data acquired using acceleration sensors contacting an anatomical body part of a patient. The anatomical body part may be a patient's head, but may also be any other anatomical body part. The method comprises executing, on at least one processor of at least one computer (for example at least one computer being part of a portable, for example a handheld device such as a mobile phone or a tablet computer), the following exemplary steps which are executed by the at least one processor.

In a (for example first) exemplary step, acceleration measurement data is acquired which describes acceleration signals acquired using the acceleration sensors. The one or more acceleration sensors may be held in place via any device suitable for upholding contact between the sensors and the patient's anatomical body part. In case of cranial accelerometry, a headband-like structure may be used, which is fitted with a plurality of acceleration sensors such that, as the headband is worn by the patient, the plurality of sensors are placed at various desired locations on the patient's head, at which locations the sensors detect acceleration of the cranium and send corresponding signals to a computer for further processing.

In a (for example second) exemplary step, artefact data is determined based on the acceleration measurement data which describes existence of one or more artefacts in the acceleration signals. These artefacts are undesired deviations or errors within the acceleration signals, which originate from sources other than the to be measured accelerations from the region of interest, which is an anatomical body part, and can for example be the cranium. For example, artefacts may result from ambient noise such as speech, breathing noise of the patient, noises caused by acceleration sensors physically contacting objects such as the patient's pillow, working noises of persons or machines, or even acoustic signals emitted by machines. As these artefacts are of an origin different to the cranium accelerations of interest, they need to be identified and eliminated from the acceleration signals. Artefacts may for example be identified with the help of sensors adapted to measure ambient noise, for example, sound pressure level (SPL) sensors which may be provided together with the acceleration sensors, for example as part of a sensor supporting structure such as the aforementioned headband. Similarities in the measurements acquired via the one or more sound sensors and the one or more acceleration sensors may thereby indicate ambient sound induced artefacts that need to be eliminated. This may take place by excluding signal sections including the identified artefacts from further processing. In the alternative, artefacts may also be eliminated by determining and removing their waveform from the acceleration signals in the time domain. In a further alternative, a spectral analysis of the measurement signals may be followed by a spectral subtraction of the identified artefacts so as to remove the artefacts from the measurement signals. In the latter case, it is possible that the artefact bearing signal sections are considered for further processing. For example, determining artefact data involves decomposing the acquired acceleration signals into a plurality of frequency ranges and analysing the decomposed signals for identifying artefacts.

Artefacts may also be caused by structure-born sound, that may for example result from acceleration sensors contacting objects such as the patient's pillow. These artefacts may be identified via a time-domain based analysis of the acceleration signals. Sharp, spike-like artefacts may indicate such contacts, which may therefore be easily identified via a Fourier-analysis of the acceleration signals within the frequency domain into which the signal was transferred from the time domain. As soon as artefacts have been identified as such, these artefacts are either removed from the acceleration signals or the affected sections of the acceleration signals are disregarded in subsequent processing steps.

In a (for example third) exemplary step, usable signal data is determined based on the acceleration measurement data and the artefact data, wherein the usable signal data describes continuous sections of the acceleration signals which are free from identified artefacts, i.e. no acceleration signals containing artefacts are processed in subsequent steps. In other words, all of the acceleration signals described by the usable signal data are generally usable for further processing, wherein features are identified in the acceleration signals and are eventually compared with features from acceleration signals acquired from other patients/subjects having a specific physiological status. In an example, this involves acquiring further sensor data of a different type than the acceleration measurement data, particularly describing at least one of
an acoustic noise of an environmental origin, including structure-borne and/or airborne sound; and
a heart rate of the patient;

wherein, based at least in part on the additional sensor data, the artefacts are identified and/or the continuous sections are divided into segments.

In a (for example fourth) exemplary step, feature data is determined based on the usable signal data, which describes at least one predefined feature in the acceleration signals. In other words, unique features are identified in the acceleration signals, wherein identification may be based on known properties of features which may be stored in a database. Such features may be identified within the time-domain of the acceleration signals, within time-frequency representations, and/or within the frequency-domain of the acceleration signals and/or in a statistical domain of the acceleration signals. Features may be identified in the acceleration signals received from at least one acceleration sensor, particularly received from a plurality of acceleration sensors. Acceleration signals received from a plurality of sensors may even undergo a combined consideration so as to identify differential features amongst these signals. For example, determining feature data involves determining signal component data based on the usable signal data and/or the segmented signal data, which describes at least one of the continuous sections and/or at least one of the segments thereof decomposed into a plurality of frequency ranges.

In a (for example fifth) exemplary step, reference feature data is determined, which describes at least one predefined feature in acceleration signals acquired for at least one reference subject of a specific medical condition. In other words, acceleration signals which may have been previously acquired from one or more, preferably a plurality of reference subjects and may have been recorded and stored in a database are compared with each other so as to determine similarities or dissimilarities in regards to unique features identified within the respective acceleration signals. It should be appreciated that the measurements taken from the respective subjects need to be sufficiently similar in how they are acquired so as to allow for a comparison amongst them as well as a meaningful deduction of similarities and dissimilarities regarding the features contained in the compared signals.

In a (for example sixth) exemplary step, classification data is determined based on the feature data and the reference feature data, which describes a grade of similarity between the at least one predefined feature in the acceleration signals acquired for the patient and the at least one predefined feature in the acceleration signals acquired for the at least one reference subject. For example, the features identified in the acceleration signals acquired from the patient and the at least one reference subject are compared with each other to find similarities or dissimilarities in the respective acceleration signals. Based on such comparison, the present patient can be compared with a sample of reference subjects, wherein any approach of using thresholds, confidence intervals, clustering, registration of features, or any feasible combination thereof may be considered to classify the present patient with respect to the number of reference subjects.

For example, determining classification data involves defining one or more classes, each class including at least one reference subject, and defining at least one threshold for the at least one predefined feature in the acceleration signals acquired for the patient, wherein an affiliation of the patient to the one or more classes is based on whether or not the at least one predefined feature in the acceleration signals acquired for the patient surpasses the at least one threshold. A threshold-based approach may involve determining a class for the patient by defining a predetermined threshold which is defined on the basis of one or more reference subjects that have been assigned to this specific class. Depending on whether or not the subject's at least one feature surpasses the predetermined threshold, the subject is either assigned to this specific class or not.

In a further example, confidence intervals may be used for classification. For the one or more features extracted from the acceleration measurements of the patient, it is determined whether it is inside or outside a predefined confidence interval. In case the one or more features is outside a confidence interval for a specific class, the feature or even the patient is assigned to a different class.

In a still further example, determining classification data involves defining one of more clusters in a space having two or more dimensions, the one or more clusters being defined by accumulations of predefined features in the acceleration signals acquired for a plurality of reference subjects, wherein an affiliation of the patient to the one or more clusters is based on the distance between the at least one predefined feature and the one or more clusters in the space having two or more dimensions. Cluster analysis or clustering may be used for classification, wherein clusters are assigned to accumulations of predefined features in a multi-dimensional, for example 2D or 3D space. Within each cluster, a cluster-center is determined, for example via K-means clustering. Consequently, the classification may be based on determining the distance between the one or more features determined for the patient and the respective cluster-centers, such that the feature or even the patient is assigned to the closest cluster.

In a still further example, determining classification data involves performing a regression analysis, for example a linear regression or a sigmoidal regression. The regression analysis may also involve the use of thresholds so as to implement binary classifications.

For example, the regression analysis is used for predictive modelling, in which an algorithm is used to predict continuous outcomes such as medical scorings, infarct sizes, etc. For this, the algorithm is trained to understand the relationship between independent variables (e.g., medical scorings, infarct sizes, etc.) of known data points and their respective outcome (e.g., stroke type, patient outcome, etc). The trained model is then used to predict the outcome of actual regression data features. As will be described in more detail below, the feature space containing the features which have been extracted from the acceleration signals can be extended with further features extracted from any other feasible measurements on the patient or any other data sources.

Regarding the acceleration signals as well as the signals/data received from other data sources, any of the features may be extracted from original signals (e.g. from the time domain of signals), from decomposed signals (e.g. from the frequency domain of signals) as well as from any relationship or comparison between these signals. Such comparison may include differences, ratios or any other feasible mathematical comparison between these signals. In this context, it should be noted that the acceleration signals may be influenced by the patient's respiration. Thus, it may be desirable to determine the correlation between the respiration cycle on the one hand and the acceleration signals on the other hand. For example, a modulation of heart related signals or information may be utilized to approximate the patient's respiration cycle. Such heart-related signals or information may be retrieved from one or more PPG-sensors, a phonocardiogram, a seismocardiogram or similar. On that basis, the phase of the respiration cycle and the acceleration signal, particularly the continuous sections or the segments derived therefrom, can be assigned to each other. In particular, the segmented signal data can be annotated with corresponding information as to the assigned respiration cycle phase for subsequent processing. Eventually, this allows for identifying and selecting segments with either the same or different respiratory phases for or during subsequent method steps described herein.

Additionally or alternatively to the above statistical approaches, machine learning approaches may also be used for classifying the patient with respect to a number of reference subjects. For example, features which have been extracted from acceleration signals or acquired from other measurements or data sources may be fed to a pretrained machine learning algorithm, which has been trained based on features derived from a sufficiently large number of reference subjects that either have a respective physiological condition or not. For example, determining classification data involves utilizing a pretrained machine learning algorithm, wherein a training set for the machine learning algorithm includes a plurality of reference subjects having the specific medical condition and a plurality of reference subjects not having the specific medical condition.

In an example of the method according to the first aspect, determining usable signal data involves determining segmented signal data based on the usable signal data, which describes the continuous sections divided into a plurality of segments. For example, the sections which are free of identified artefacts are partitioned or divided into smaller, i.e. shorter (sub-)segments. The partitioning may be based on a heartbeat signal which is for example detected via one or more heartbeat sensors, such as photoplethysmography (PPG) sensors. In the alternative, the heartbeat can also be detected within the acceleration signals by detecting characteristic repetitive waveforms within the acceleration signals. In such case, the heartbeat can be detected directly via the signals acquired from the one or more acceleration sensors, as is for example described in US 2018/0296107, or for example by filtering for repetitive waveforms in the time series signal describing the heartbeat.

By doing so, the continuous, artefact-free sections may be divided into segments which may have a length of at least one cardiac cycle. It is also possible that each cardiac cycle is divided into a plurality of segments which for example cover a systolic and/or diastolic section of the heartbeat. Further, the segments may cover a substantially equal length of time. In a specific example, the segments each cover substantially one cardiac cycle.

In a further example, the segments covering one or more cardiac cycles are searched for additional artefacts which have not been described in the artefact data and have therefore not been removed, or signal sections containing these artefacts have not been left unconsidered so far. In case any further artefacts are identified within the heartbeat-segmented signals, the corresponding segments can be left unconsidered for further processing. Identification of artefacts may be based on the same and/or other principles that have been implemented for the artefact-identification described further above with respect to the unsegmented acceleration signals. It needs to be noted here that arrhythmic and/or ectopic heartbeats may occur under normal as well as under pathophysiological conditions of the patient. As the cranial acceleration signals are sensitive to such arrhythmic and/or ectopic heartbeats, this has impact on the signal characteristics. Thus, in a further example, these signal characteristics may be identified via cranial acceleration features such as the signal amplitude. Furthermore, ratios and statistical features such as the skewness on a heartbeat-by-heartbeat basis of the aforementioned features allows for identifying arrhythmic and/or ectopic heartbeats. Moreover, it is possible to implement thresholds which may for example be determined empirically, for identifying arrhythmic and/or ectopic heartbeats. Eventually, arrhythmic and/or ectopic heartbeats can be identified and in particular annotated for or during subsequent method steps described herein.

In a further example, determining usable signal data involves generating, based on the quantity and/or quality of identified artefacts, an indicator describing usability of the acquired acceleration signals, particularly usability of the continuous sections and/or of the segments thereof, for being processed for determining feature data, particularly wherein acceleration signals, particularly continuous sections and/or of segments thereof, are disregarded for determining feature data when the acquired acceleration signals, particularly usability of the continuous sections and/or of the segments thereof, are indicated as not usable.

In other words, a tolerance range may be defined in which continuous sections of the acceleration signals and/or partitioned segments thereof are considered usable for further processing even tough artefacts have been discovered therein, as long as these artefacts are considered not harmful for further processing, i.e. for detecting, identifying and evaluating features within the acceleration signals.

As already mentioned further above, additional features may be considered which do not derive from acceleration signals, but from other measurements on the patient or from any other feasible data source. For example, determining feature data may involve acquiring condition data, particularly condition data input manually by a user, relating to a condition of the patient, particularly a medical and/or pathological condition of the patient. For example, such features can be acquired via a user input, for example describing age, gender, height, weight, head circumference, pre-existing medical conditions, scoring obtained by clinically accepted evaluation methods such as the National Institutes of Health, Stroke, Scale (NIHSS), Face-Arms-Speech-Time (FAST) tests, the Cincinnati Prehospital Stroke Scale Test, or any other officially accepted score for evaluating the severity of a suspected stroke.

In a further example, determining condition data may describe at least one of:
a motoric deficiency of the patient, particularly detected via a handheld device grasped by the patient;
a gaze or a visual impairment of the patient, particularly detected via a device tracking at least one of the patient's eyes;
a property of the patient's peripheral vasculature;
an activity of the brain or the heart of the patient;
a blood flow of the patient.

For example, any of the above features may be provided using additional devices, such as:
a handheld device for finger pressure/force measurement to determine parameters for features related to motor deficits;
an eye tracking device to monitor gaze or visual impairments;
a device for taking measurements on the vasculature, such as blood flow, blood velocity or similar;
physiological signal measuring devices for monitoring brain activity (e.g. electroencephalography (EEG) sensors), for monitoring heart activity (e.g. electrocardiogramaensors), or monitoring blood flow (e.g. doppler ultrasound sensors).

In a further example, a statistical analysis of cranial acceleration signals may reveal correlations between features extracted from segmented acceleration signals and other factors such as the age and the heart rate of the patient. Based on this information, which may be obtained from one or more previously acquired datasets, assigning and weighing of cranial acceleration features may be implemented for or during subsequent method steps described herein.

In a second aspect, the invention is directed to a computer program comprising instructions which, when the program is executed by at least one computer, causes the at least one computer to carry out method according to the first aspect. The invention may alternatively or additionally relate to a (physical, for example electrical, for example technically generated) signal wave, for example a digital signal wave, such as an electromagnetic carrier wave carrying information which represents the program, for example the aforementioned program, which for example comprises code means which are adapted to perform any or all of the steps of the method according to the first aspect. The signal wave is in one example a data carrier signal carrying the aforementioned computer program. A computer program stored on a disc is a data file, and when the file is read out and transmitted it becomes a data stream for example in the form of a (physical, for example electrical, for example technically generated) signal. The signal can be implemented as the signal wave, for example as the electromagnetic carrier wave which is described herein. For example, the signal, for example the signal wave is constituted to be transmitted via a computer network, for example LAN, WLAN, WAN, mobile network, for example the internet. For example, the signal, for example the signal wave, is constituted to be transmitted by optic or acoustic data transmission. The invention according to the second aspect therefore may alternatively or additionally relate to a data stream representative of the aforementioned program, i.e. comprising the program.

In a third aspect, the invention is directed to a computer-readable storage medium on which the program according to the second aspect is stored. The program storage medium is for example non-transitory.

In a fourth aspect, the invention is directed to at least one computer (for example, a computer), comprising at least one processor (for example, a processor), wherein the program according to the second aspect is executed by the processor, or wherein the at least one computer comprises the computer-readable storage medium according to the third aspect.

In a fifth aspect, the invention is directed to a medical system (for example a system for cranial accelerometry), comprising:
a) the at least one computer according to the fourth aspect;
b) at least one electronic data storage device storing at least the acceleration measurement data; and
c) at least one acceleration sensor for receiving acceleration signals from an anatomical body part;
d) for example, at least one heartbeat detector for receiving the patient's heartbeat signals;
   wherein the at least one computer is operably coupled to
the at least one electronic data storage device for acquiring, from the at least one data storage device, at least the acceleration measurement data, and
the acceleration sensor, for receiving, from the acceleration sensor, the patient's acceleration signals and generating, from the acceleration signals, acceleration measurement data, and
for example, the heartbeat detector, for receiving, from the heartbeat detector, the patient's heartbeat signals and generating, from the heartbeat signals, heartbeat signal data.

Alternatively or additionally, the invention according to the fifth aspect is directed to a for example non-transitory computer-readable program storage medium storing a program for causing the computer according to the fourth aspect to execute the data processing steps of the method according to the first aspect.

For example, the invention does not involve or in particular comprise or encompass an invasive step which would represent a substantial physical interference with the body requiring professional medical expertise to be carried out and entailing a substantial health risk even when carried out with the required professional care and expertise.

### DEFINITIONS

In this section, definitions for specific terminology used in this disclosure are offered which also form part of the present disclosure.

The method in accordance with the invention is for example a computer-implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n-and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term computer includes a server resource. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a computer program comprising instructions which, when on the program is executed by a computer, cause the computer to carry out the method or methods, for example, the steps of the method or methods, described herein and/or to a computer-readable storage medium (for example, a non-transitory computer-readable storage medium) on which the program is stored and/or to a computer comprising said program storage medium and/or to a (physical, for example electrical, for example technically generated) signal wave, for example a digital signal wave, such as an electromagnetic carrier wave carrying information which represents the program, for example the aforementioned program, which for example comprises code means which are adapted to perform any or all of the method steps described herein. The signal wave is in one example a data carrier signal carrying the aforementioned computer program. The invention also relates to a computer comprising at least one processor and/or the aforementioned computer-readable storage medium and for example a memory, wherein the program is executed by the processor.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

The expression “acquiring data” for example encompasses (within the framework of a computer implemented method) the scenario in which the data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. A step of “determining” as described herein for example comprises or consists of issuing a command to perform the determination described herein. For example, the step comprises or consists of issuing a command to cause a computer, for example a remote computer, for example a remote server, for example in the cloud, to perform the determination. Alternatively or additionally, a step of “determination” as described herein for example comprises or consists of receiving the data resulting from the determination described herein, for example receiving the resulting data from the remote computer, for example from that remote computer which has been caused to perform the determination. The meaning of “acquiring data” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates the basic steps of the method according to the first aspect;

FIG. 2 shows an embodiment of the present invention, specifically the method according to the first aspect;

FIGS. 3 and 4 show a flow diagram illustrating the process of eliminating artefacts from acceleration signals;

FIG. 5 gives an overview of a data processing flow in which the method according to the present invention can be used;

FIG. 6 is a schematic illustration of the system according to the fifth aspect; and

FIG. 7 shows an example of recorded acceleration measurements.

### DESCRIPTION OF EMBODIMENTS

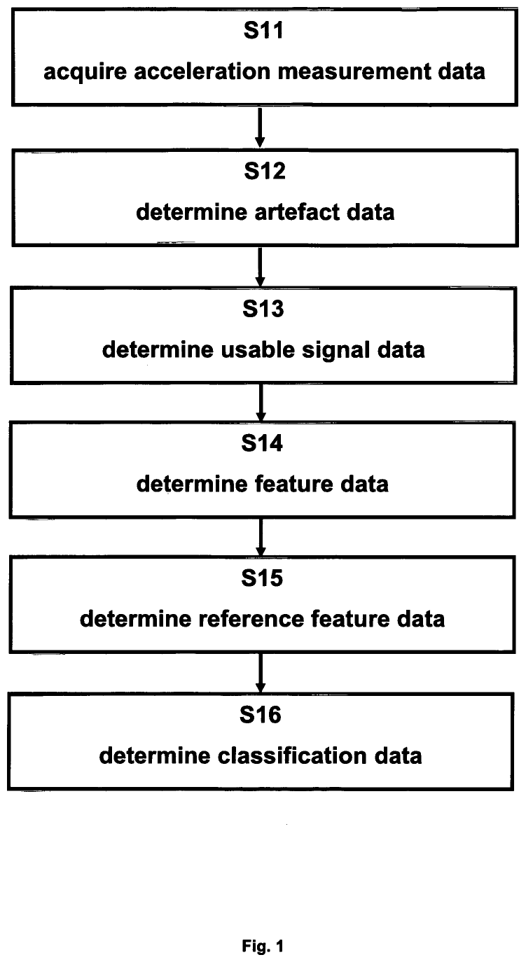

FIG. 1 illustrates the basic steps of the method according to the first aspect, in which step S11 encompasses acquisition of the acceleration measurement data, step S12 encompasses determination of the artefact data, step S13 encompasses. determination of the usable signal data, step 14 encompasses determination of the feature data, step 15 encompasses determination of the reference feature data, and step 16 encompasses determination of the classification data.

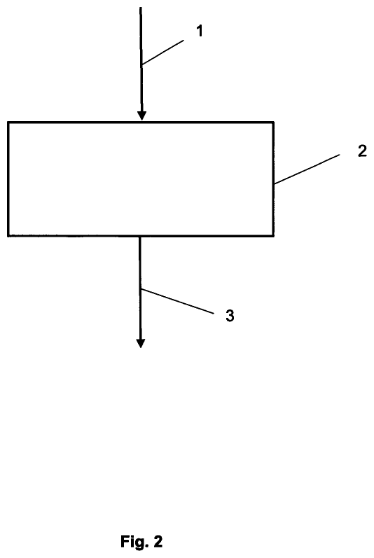

FIG. 2 illustrates an embodiment of the present invention that includes all essential features of the invention. In this embodiment, the entire data processing which is part of the method according to the first aspect is performed by a computer 2. Reference sign 1 denotes the input of data acquired by the method according to the first aspect into the computer 2 and reference sign 3 denotes the output of data determined by the method according to the first aspect.

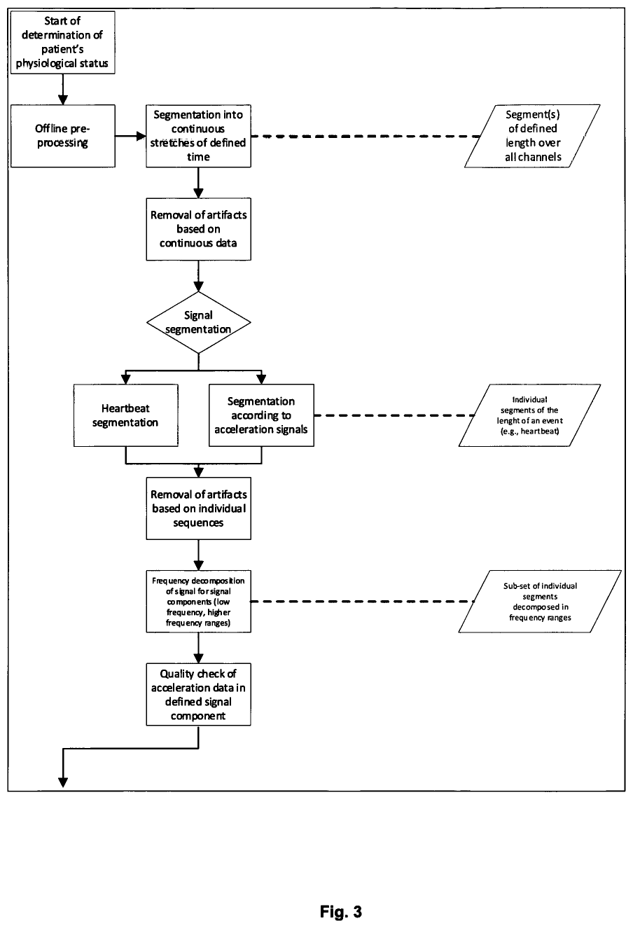

FIGS. 3 and 4 illustrate the flow of performing cranial accelerometry on a patient, removing artefacts from the acceleration signals, and extracting and evaluating features of the artefact-free acceleration signals. Determination of the patient's physiological status is started with an offline processing of acceleration signals, i.e. processing previously-acquired acceleration signals acquired via at least one, particularly via a plurality of acceleration sensors contacting the patient's region of interest, e.g. the cranium. An individual signal channel is thereby assigned to each one of the acceleration sensors. The acquired acceleration signals are segmented into continuous stretches of a sufficient time length, i.e. of at least a predefined time length, so as to deliver sufficient data for the processing steps that are to follow. As an optional step, the continuous stretches may be correlated with and/or rated on the basis of corresponding respiration cycle phases derived from heartbeat-related signals. Artefacts identified in the acceleration signals are then either removed from the acceleration signals or signal sections containing these artefacts are “cut out” and discarded, i.e. left unconsidered in the subsequent processing steps. The now artefact free sections of the acceleration signals are then segmented/divided into a plurality of segments of a predefined length. In the shown example, the segment length is substantially equal to a cardiac cycle. Thus, the segmentation may either be based on signal data describing the heartbeat, or “directly” on the acceleration signals by detecting characteristic repetitive waveforms in the acceleration signals. The segments of a substantially equal length of time now undergo an additional search for artefacts. Segments which contain detected artefacts are left unconsidered in the subsequent processing steps. As an optional step, segments containing one or more of arrhythmic heartbeats, ectopic heartbeats and premature ventricular contractions may also be removed. In order to search the remaining artefact free sections of the acceleration signals for unique features, these sections are decomposed, i.e. converted from the time domain into the frequency domain, particularly into a) low-frequency (e.g. 0.5-20 Hz) and b) higher frequencies (e.g. 20 Hz to Nyquist frequency). The decomposed signal segments are then subjected to a quality check so as to determine whether the decomposed signal segments are of a sufficient quality for feature extraction and evaluation. If it is determined that a sufficient amount of segmented and decomposed acceleration signal segments of a sufficient quality is available, feature detection and evaluation can commence (cf. FIG. 4). While patient positioning may be estimated during recording and parameters such as arterial stiffness, intra-cranial pressure (ICP), blood pressure etc. can be extracted for feature augmentation and feature rating/selection, features are detected within the segmented and decomposed acceleration signals. Based on confounding factors such as age and heart rate of the patient, the cranial acceleration features may, as an optional step, be identified and/or weighted. Knowledge of certain properties of features of interest help in identifying such features in the (segmented and decomposed) acceleration signals. This may be either done via a statistical approach or via a machine learning approach, particularly wherein a list of predictive features is provided for each channel. The extracted features are then classified by implementing one of the classification approaches described further above. The features extracted from the acceleration signals and additional features acquired from other measurement sources and/or data sources are enhanced before the patient is classified within the number of reference subjects. A report on the classification results is then output.

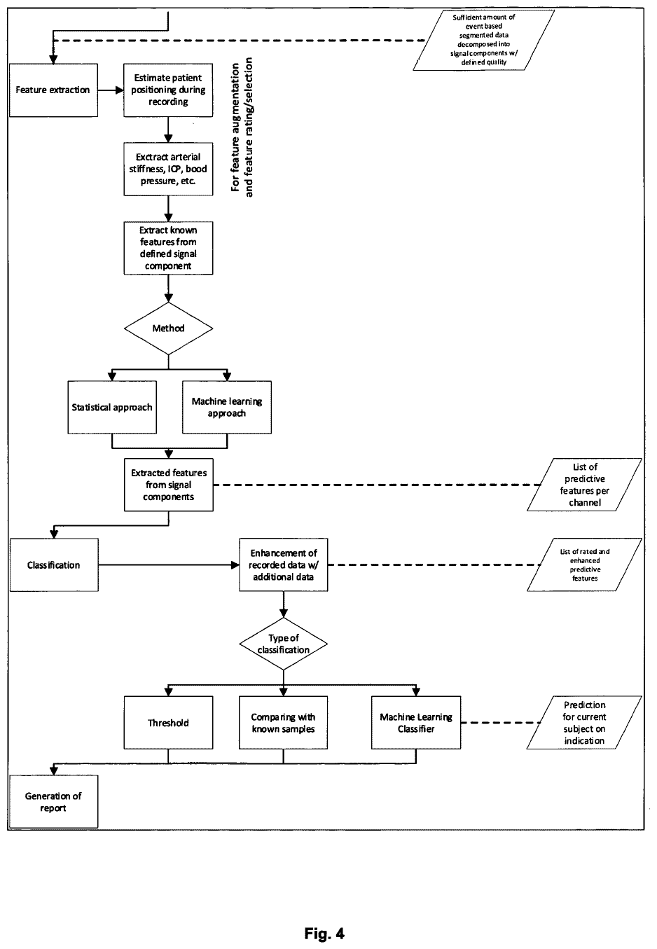

FIG. 5 illustrates a complete workflow which lies within the scope of the disclosed invention and includes steps which are performed previously and subsequently to the above-describes data processing. After placing the headband/headset on the patient and conducting a pre-check and online pre-processing as described in PCT/EP2021/078408, artefacts are detected and removed from the acceleration measurement data. The acceleration signals may be segmented into a part indicating a heartbeat signal and a part indicating another acceleration-induced signal component, which may be followed by signal decomposition according to frequency and optionally feature augmentation. Subsequently, an accordingly configured machine learning classifier or a statistical approach is used to determine, on the basis of the result of the foregoing data processing, the patient's physiological status. Optionally, a report about the results and for example the metainformation characterising the measurement is then generated.

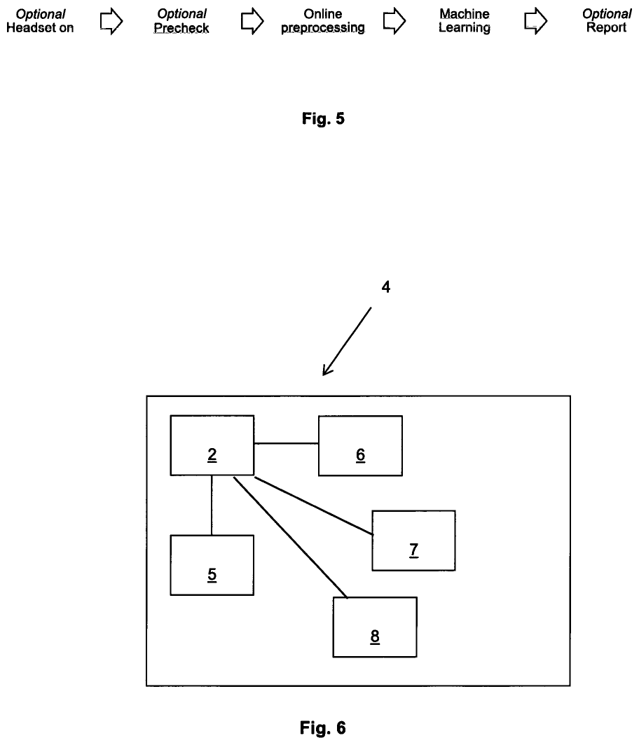

FIG. 6 is a schematic illustration of the medical system 4 according to the fifth aspect. The system is in its entirety identified by reference sign 4 and comprises a computer 5, an electronic data storage device (such as a hard disc) 6 for storing at least the patient data and a medical device 7 (such as a radiation treatment apparatus). The components of the medical system 4 have the functionalities and properties explained above with regard to the fifth aspect of this disclosure.

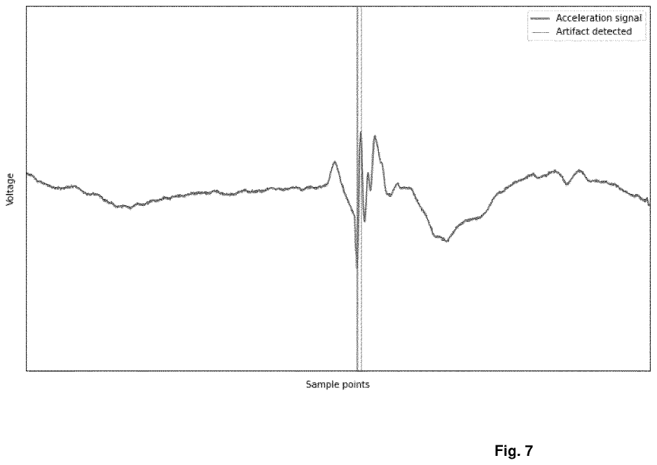

FIG. 7 shows a raw acceleration signal acquired via an acceleration sensor contacting the patient's cranium. Occasionally, spike-like artifacts are observed in the acceleration signal which can be detected by artifact-detection methods.

An additional explanation of the system lying in the scope of the present invention is presented in the following.

The system comprises various subsystem components that include:
a headset that is patient-contacting and includes:
   one or more photoplethysmography (PPG) sensors for detecting the heartbeat, heart rate, and timing;
   one or more sound pressure level (SPL) sensors for detecting ambient environment noise; and
   a plurality of accelerometer sensors to detect the acceleration at dedicated locations around the patient's head.
a data collector which digitizes the analogue sensor signals (either as an individual component or integrated into the sensor elements);
a computer unit which incorporates the device software and space to store the recording data; and
device software, which provides a user interface, hardware control, software libraries, and algorithms for signal preparation, signal processing, signal separation and classification for a plurality of clinical indications.

The system collects and stores sensor data caused by, for example the pulsatile blood flow from the cardiac cycle, leading to a slight acceleration of the skull. The system uses for example piezoelectric-based accelerometer sensors that measure a variety of signal components originating from the response of the head/brain to the blood flow.

The plurality of acceleration sensors sense the motion and the data collector digitizes the signal. The computer unit provides the user interface, stores the data, performs the signal separation and the classification of the recorded data specific to the clinical indication.

A user places the headset of the device on a patient and sets up the user interface to perform a recording. Typically, the user will perform a recording that is approximately one to two minutes long, though in some cases, if the patient moves or displaces the headset, the recording may be prolonged. The device software analyzes the recording and separates the recorded signal into its signal components. Predictive features are calculated for each signal components and these are then classified by statistical and/or machine learning approaches using known thresholds, data of known conditions, etc. The device software then displays the result of classification defined clinical indications to the user. preferred that the recording is mostly free of artefacts which could interfere with example classification. Disclosed are methods for quality analysis of the signal before the recording starts and continuous quality check during a recording.
The headset is mounted on the subject's head and the device is switched on. A recording is performed and once the recording is finished, it is evaluated by utilizing the following preprocessing approach:
   Artefact detection and/or removal
      After a recording, the recorded data is preprocessed to remove sections containing artefacts from the recording or to remove artefacts from the respective sections and thus restoring the original waveforms without artefacts. The artefacts may be due to the following:
         Acceleration Signal Quality Check
         Background noise and sounds. Background noise and sounds (speech, beeping of vital monitors, breathing of the subject etc.) are detected by determining a measure of similarity between the SPL sensor and the plurality of acceleration sensors (e.g. coherence analysis). The measure of similarity with the SPL channel is determined for each acceleration channel individually. Furthermore, the measure of similarity can also be achieved by signal decomposition using decomposition methods (e.g. principle component analysis, independent component analysis, blind source separation, spectral subtraction, adaptative noise canceling, noise modeling via convolutional neural networks etc.). If background noise is detected the respective sections are either excluded from the analysis or the signal waveform without artefacts is restored by detecting and removing the artefact waveform in the time domain or the artefact energy via spectral analyzes and subsequent spectral subtraction. In the latter case, the artefacts are removed and segments will be used for analysis.
         Contact of headset sensors with objects during measurement (e.g., pillow for stabilizing subjects head, head rest, etc.). A time-domain based analysis may be utilized to detect short spike-like artefacts. To do so, a signal is decomposed using decomposition methods (e.g. Fourier-decomposition). Next, a description of a sample point range (e.g. a running median, determined by a window size) on the resulting signal is determined. Both the decomposed signal and the description of the sample point range are compared against each other sample point-wise (e.g. via subtraction) and tagged as an artefact if a defined threshold is exceeded.
Creating a sub-set of good segments
   The above-mentioned preprocessing methods alone or in combination with the results of the online preprocessing methods as described in PCT/EP2021/078408, which is incorporated herein by reference, may be used to create a sub-set of the recorded sensor data containing data, that meets all quality related criteria.
Heartbeat segmentation
   The segments without artefacts are next processed and further subsegmented by identification of individual heartbeats.
   The heartbeat signal is detected using for example a heartbeat sensor (e.g. a PPG sensor) or a plurality of heartbeat sensors.
   The heartbeat can also be detected using characteristic repetitive waveforms within the acceleration signal. In some cases heartbeats can be detected directly via the acceleration sensor.
Frequency decomposition
   The individual heartbeats are decomposed to isolate physiological components within the accelerometer signal reflecting low, medium, and high frequency using methods such as Fourier decomposition, forward-backward filtering, and spectral subtraction.
Feature extraction
   Features from the time domain, the frequency domain, and the statistical domain are extracted for each acceleration channel, for each frequency range and for sensor combinations (to derive differential features).
Feature augmentation
   Further features will be derived from sensors other than the acceleration sensors, other devices, or user input for example:
      Features are for example extracted from the heartbeat sensor or plurality of heartbeat sensors. Features are for example risk factors of the respective condition such as arterial stiffness, intracranial pressure, blood pressure, atrial fibrillation.
      Features can be provided via user input into the software (e.g. age, gender, pre-existing conditions, scoring obtained by clinically accepted evaluation methods such as NIHSS, FAST, Cincinnati Prehospital Stroke Scale, etc.). The scores are officially accepted values used for evaluating the severity of a suspected stroke and well described in the literature. They do not represent a real measurement value but are based on subjective evaluation of the patient by a medically trained person.
      Features can be provided using additional devices for example:
         Eye tracking can be used for example to check for gaze or visual impairments on one or both sides giving an additional parameter indicating the location of the physiological impairment of the patient
         A device for determining features of the peripheral vasculature (e.g. blood flow, velocity) can be used. Measurement of the vasculature can also provide a normalization of the flow to the brain of individual subjects, leading to reduction of inter-subject variability.
         Devices for collecting patient physiological signals to add for example further features to the evaluation, for example brain activity (EEG), heart activity (ECG), blood flow (doppler sonography)
Classification with statistical methods
   After extraction of features, the features of a given subjects can be compared to a sample of control subjects and subjects with the respective condition e.g. using thresholds, confidence intervals, clustering, regression of features).
      Threshold-based methods determine a class (i.e. on a binary classification: class 0 or class 1) by using a predetermined threshold based on samples of the classes. Depending whether the subject surpasses a threshold or not, the subject is assigned to the respective class.
      Confidence intervals are a statistical measure which described a precision interval of a given feature. When used for classification the feature from a test subject can e.g. be outside of an a priori defined confidence interval for class 0 and therefore assigned to class 1.
      Clusters are assigned to accumulations of given features in the 2D/3D space. Within each cluster, a centroid (“mid point”) of the cluster is determined (e.g. with K-means clustering). Clusters can be used for classification by calculating which centroid is closer to a test subject and assign a subject accordingly.
      Features can be described by regression (e.g. linear, sigmoidal). For binary classifications, a threshold on the regression can be used to assign the individual class.
   Feature augmentation can be used to extend the feature space for classification
   Comparing features extracted from original, decomposed signal, or signal relationships (e.g. ratios, differences) and compare these by statistical means
Classification with machine learning methods
   After extraction of features, a pretrained machine learning algorithm may be employed for a given recording.
      The training set for machine learning algorithm will be a sufficiently large dataset containing subjects with and without the respective condition.
      Feature augmentation will be used to extend the feature space for classification.
Devices contained in the system are for example:
   a headset containing:
      one or more PPG sensors
      one or more SPL sensors
      a plurality of acceleration sensors
   a data collector
   a computer unit
   a device software
   a device for determining vascular features of the subject
   a device for eye tracking of the subject.

1. A computer-implemented medical method of processing data acquired using acceleration sensors contacting an anatomical body part of a patient, the method comprising:
acquiring acceleration measurement data that describes acceleration signals acquired using the acceleration sensors;
determining artefact data based on the acceleration measurement data, wherein the artefact data describes existence of one or more artefacts in the acceleration signals;
determining usable signal data based on the acceleration measurement data and the artefact data, wherein the usable signal data describes continuous sections of the acceleration signals which are free of identified artefacts;
determining feature data based on the usable signal data, wherein the feature data describes at least one predefined feature in the acceleration signals;
determining reference feature data that describes at least one predefined feature in acceleration signals acquired for at least one reference subject of a specific medical condition; and
determining classification data based on the feature data and the reference feature data, wherein the classification data describes a grade of similarity between the at least one predefined feature in the acceleration signals acquired for the patient and the at least one predefined feature in the acceleration signals acquired for the at least one reference subject.

2. The method according to claim 1, wherein the determining the usable signal data comprises determining segmented signal data based on the usable signal data, wherein the segmented signal data describes the continuous sections divided into a plurality of segments.

3. The method according to claim 2, wherein the segmented signal data describes continuous sections divided into segments having a length of at least one cardiac cycle, wherein the segments cover a substantially equal length of time.

4. The method according to claim 2, further comprising searching the segments for additional artefacts not described in the artefact data.

5. The method according to claim 3, wherein the determining the usable signal data comprises generating, based on the quantity and/or quality of identified artefacts, an indicator describing usability of the continuous sections and/or of the segments thereof, for being processed for determining feature data, wherein continuous sections and/or of segments thereof, are disregarded for determining feature data when the usability of the continuous sections and/or of the segments thereof, are indicated as not usable.

6. The method according to claim 1, wherein the determining the usable signal data comprises acquiring additional sensor data of a different type than the acceleration measurement data that describes at least one of:
an acoustic noise of an environmental origin, comprising structure-borne and/or airborne sound; and
a heart rate of the patient;
wherein, based at least in part on the additional sensor data, the artefacts are identified and/or the continuous sections are divided into segments.

7. The method according to claim 1, wherein the determining the feature data comprises acquiring condition data input manually by a user, relating to the patient and/or a medical and/or pathological condition of the patient.

8. The method according to claim 7, wherein the determining the condition data describes at least one of:
a motoric deficiency of the patient acquired via a handheld device grasped by the patient;
a gaze or a visual impairment of the patient acquired via a device tracking at least one of the patient's eyes;
a property of the patient's peripheral vasculature;
an activity of the brain or the heart of the patient; and/or
a blood flow of the patient.

9. The method according to claim 1, wherein the determining the artefact data comprises decomposing the acquired acceleration signals into a plurality of frequency ranges and analyzing the decomposed signals for identifying artefacts.

10. The method according to claim 3, wherein the determining the feature data comprises determining signal component data based on the usable signal data and/or the segmented signal data, that describes at least one of the continuous sections and/or at least one of the segments thereof decomposed into a plurality of frequency ranges.

11. The method according to claim 1, wherein the determining the classification data comprises defining one or more classes, each class including at least one reference subject, and defining at least one threshold for the at least one predefined feature in the acceleration signals acquired for the patient, wherein an affiliation of the patient to the one or more classes is based on whether or not the at least one predefined feature in the acceleration signals acquired for the patient surpasses the at least one threshold.

12. The method according to claim 1, wherein the determining the classification data comprises defining one of more clusters in a space having two or more dimensions, the one or more clusters being defined by accumulations of predefined features in the acceleration signals acquired for a plurality of reference subjects, wherein an affiliation of the patient to the one or more clusters is based on the distance between the at least one predefined feature and the one or more clusters in the space having two or more dimensions.

13. The method according to claim 1, wherein the determining the classification data comprises utilizing a pretrained machine learning algorithm, wherein a training set for the machine learning algorithm comprises a plurality of reference subjects having the specific medical condition and a plurality of reference subjects not having the specific medical condition.

14. A non-transitory computer-readable storage medium storing instructions thereon that, when the instructions are executed by a processor device of an associated computer, cause the computer to perform a method of processing data acquired using acceleration sensors contacting an anatomical body part of a patient, by:
acquiring acceleration measurement data that describes acceleration signals acquired using the acceleration sensors;
determining artefact data based on the acceleration measurement data, wherein the artefact data describes existence of one or more artefacts in the acceleration signals;
determining usable signal data based on the acceleration measurement data and the artefact data, wherein the usable signal data describes continuous sections of the acceleration signals which are free of identified artefacts;
determining feature data based on the usable signal data, wherein the feature data describes at least one predefined feature in the acceleration signals;
determining reference feature data that describes at least one predefined feature in acceleration signals acquired for at least one reference subject of a specific medical condition; and
determining classification data based on the feature data and the reference feature data, wherein the classification data describes a grade of similarity between the at least one predefined feature in the acceleration signals acquired for the patient and the at least one predefined feature in the acceleration signals acquired for the at least one reference subject.

15. A medical system comprising:
at least one computer comprising a processor and a non-transient computer-readable storage medium storing instructions thereon that are executable by the processor;
at least one electronic data storage device storing at least the reference feature data; and
a medical device for acquiring acceleration measurement data of a patient, including acceleration sensors contacting an anatomical body part of the patient,
wherein the at least one computer is operably coupled with:
the at least one electronic data storage device for acquiring, from the at least one data storage device, at least the reference feature data, and
the medical device for acquiring, from the medical device, at least the acceleration measurement data,
wherein the system is operative to:
determine artefact data based on the acceleration measurement data, wherein the artefact data describes existence of one or more artefacts in the acceleration signals;
determine usable signal data based on the acceleration measurement data and the artefact data, wherein the usable signal data describes continuous sections of the acceleration signals which are free of identified artefacts;
determine feature data based on the usable signal data, wherein the feature data describes at least one predefined feature in the acceleration signals;
determine reference feature data that describes at least one predefined feature in acceleration signals acquired for at least one reference subject of a specific medical condition; and
determine classification data based on the feature data and the reference feature data, wherein the classification data describes a grade of similarity between the at least one predefined feature in the acceleration signals acquired for the patient and the at least one predefined feature in the acceleration signals acquired for the at least one reference subject

16. The method according to claim 1, further comprising:
wherein the determining the usable signal data comprises determining segmented signal data based on the usable signal data and a detected heartbeat of the patient that describes the continuous sections divided into a plurality of segments having a length of at least one cardiac cycle and covering a substantial substantially equal length of time; and
generating an indicator, based on the quantity and/or quality of identified artefacts, wherein the indicator describes usability of the segments of the acquired acceleration signals for being processed for determining feature data is generated.
